SEQUENCE LISTING (24 sequences)

SEQ ID NO: 1 - protein, 64 aa, Homo sapiens
MRVLYLLFSFLFIFLMPLPGVFGGIGDPVTCLKSGAICHPVFCPRRYKQIGTCGLPGTKCCKKP

SEQ ID NO: 2 - protein, 72 aa, Homo sapiens
MQRLVLLLAISLLLYQDLPVRSEFELDRICGYGTARCRKKCRSQEYRIGRCPNTYACCLRKWDESLLNRTKP

SEQ ID NO: 3 - protein, 87 aa, Homo sapiens
MRLHLLLLILLLFSILLSPVRGGLGPAEGHCLNLFGVCRTDVCNIVEDQIGACRRRMKCCRAWWILMPIPTPLIMSDYQEPLKPNLK

SEQ ID NO: 4 - protein, 37 aa, Homo sapiens
LLGDFFRKSKEKIGKEFKRIVQRIKDFLRNLVPRTES

SEQ ID NO: 5 - DNA, 20 bp, Artificial Sequence
  Description of Artificial Sequence Synthetic primer
cccagttcctgaaatcctga

SEQ ID NO: 6 - DNA, 20 bp, Artificial Sequence
  Description of Artificial Sequence Synthetic primer
caggtgccttgaattttggt

SEQ ID NO: 7 - DNA, 20 bp, Artificial Sequence
  Description of Artificial Sequence Synthetic primer
catcagccatgagggtcttg

SEQ ID NO: 8 - DNA, 20 bp, Artificial Sequence
  Description of Artificial Sequence Synthetic primer
ggctttttgcagcattttgt

SEQ ID NO: 9 - DNA, 21 bp, Artificial Sequence
  Description of Artificial Sequence Synthetic primer
agcctagcagctatgaggatc

SEQ ID NO: 10 - DNA, 21 bp, Artificial Sequence
  Description of Artificial Sequence Synthetic primer
cttcggcagcattttgcgcca

SEQ ID NO: 11 - DNA, 20 bp, Artificial Sequence
  Description of Artificial Sequence Synthetic primer
ttccaggtgtttttggtggt

SEQ ID NO: 12 - DNA, 20 bp, Artificial Sequence
  Description of Artificial Sequence Synthetic primer
gagaccacaggtgccaattt

SEQ ID NO: 13 - DNA, 20 bp, Artificial Sequence
  Description of Artificial Sequence Synthetic primer
tccatcaggtgagtttgctg

SEQ ID NO: 14 - DNA, 20 bp, Artificial Sequence
  Description of Artificial Sequence Synthetic primer
gttcagcctgcaatttccat

SEQ ID NO: 15 - DNA, 18 bp, Artificial Sequence
  Description of Artificial Sequence Synthetic primer
ccccagccaagaatgcat

SEQ ID NO: 16 - DNA, 21 bp, Artificial Sequence
  Description of Artificial Sequence Synthetic primer
tcatttttcccgcaattgttc

SEQ ID NO: 17 - DNA, 23 bp, Artificial Sequence
  Description of Artificial Sequence Synthetic primer
caagttctaccagccaggggcaa

SEQ ID NO: 18 - DNA, 22 bp, Artificial Sequence
  Description of Artificial Sequence Synthetic primer
ttggttgatgccccagaggcag

SEQ ID NO: 19 - DNA, 21 bp, Artificial Sequence
  Description of Artificial Sequence Synthetic primer
aggtggtttgggtcctgcgga

SEQ ID NO: 20 - DNA, 24 bp, Artificial Sequence
  Description of Artificial Sequence Synthetic primer
tccaccatgctctacagcacttca

SEQ ID NO: 21 - DNA, 20 bp, Artificial Sequence
  Description of Artificial Sequence Synthetic primer
tgcattccatccaatgaaga

SEQ ID NO: 22 - DNA, 20 bp, Artificial Sequence
  Description of Artificial Sequence Synthetic primer
gaggtctcagttccccttcc

SEQ ID NO: 23 - DNA, 20 bp, Artificial Sequence
  Description of Artificial Sequence Synthetic primer
ctagagggaggcagacatgg

SEQ ID NO: 24 - DNA, 19 bp, Artificial Sequence
  Description of Artificial Sequence Synthetic primer
aggaggcggtaaggttagc

(19) United States
(12) Patent Application Publication
(10) Pub. No.: US 2012/0288860 A1
(43) Pub. Date: Nov. 15, 2012

(54) DIFFERENTIAL GENE EXPRESSION FOR DETECTING AND/OR DIFFERENTIATING LUNG DISEASE

(75) Inventors: MONIQUE VAN HOEK, CENTREVILLE, VA (US); STEVEN NATHAN, VIENNA, VA (US)

(73) Assignee: GEORGE MASON UNIVERSITY

(21) Appl. No.: 13/469,915

(22) Filed: May 11, 2012

Related U.S. Application Data
(60) Provisional application No. 61/485,370, filed on May 12, 2011.

(51) Int. Cl. C12Q 1/68

(52) U.S. Cl. 435/6.11; 435/6.12

(57) ABSTRACT

Disclosed herein are methods, constructs, kits, and the like, which can be used for detecting and/or differentiating interstitial lung disease. For example, idiopathic pulmonary fibrosis (IPF) and nonspecific interstitial pneumonia (NSIP) can be detected and/or differentiated using at least one biomarker.

### CROSS-REFERENCE TO RELATED APPLICATION

This application claims the benefit of U.S. Provisional Application No. 61/485,370, filed May 12, 2011, which is incorporated herein by reference in its entirety.

### FIELD

The present disclosure relates to lung disease and provides methods, constructs, and related kits for diagnostic and/or therapeutic applications.

### INTRODUCTION

Interstitial lung disease (ILD) includes a broad categories of diseases, including more than 100 separate disorders. Based on the cause of the disease, ILD is classified into two groups, ILD of known cause, and ILD of unknown cause. The idiopathic interstitial pneumonias (IIPs) are an important subsets of ILDs. Both nonspecific interstitial pneumonia (NSIP) and idiopathic pulmonary fibrosis (IPF) belong to the IIPs group, while sarcoidosis is an ILD with known cause.

IPF is a progressive fibrotic lung disease with high morbidity and mortality and no effective medical therapy. Common clinical features include progressive dyspnea, dry cough, and the presence of basilar ‘velcro-like’ rales on examination, which are not specific to IPF. Other pulmonary lung fibrosis diseases, such as collagen-vascular disease, chronic hypersensitivity pneumonitis, drug reactions, etc., may exhibit similar symptoms, even similar radiographic features. Diagnosis of IPF must exclude these secondary lung fibrosis diseases. The definitive diagnosis of IPF is based on surgical biopsies and pathology diagnosis. Usual interstitial pneumonia (UIP), histopathologically shown as patchy areas of fibrosis in association with areas of normal lung architecture, is the characteristic feature of IPF [38]. Once diagnosed, the median survival time is only 2.5-5 years [39] and lung transplant remains the only hope in a small minority of these patients.

Most patients with IPF exhibit unique patterns of disease progression, characterized by a long duration of symposiums described earlier prior to diagnosis and followed by a slow progressive clinical course [40]. Two to three years after diagnosis, without evidence of infectious pneumonia, heart failure, pulmonary embolism or possibility of acute lung injury, many patients experience a sudden worsening of dyspnea with influenza-like symptoms, termed acute exacerbation of IPF (AE-IPF). High Resolution Computed Tomography (HRCT) shows bi-lateral ground-glass abnormality and/or consolidation superimposed on a usual interstitial pneumonia (UIP) pattern (bibasilar subpleural reticular, tractionbronchiectasis, and honeycomb) [36, 41]. Recent study finds that only about 5-19% of IPF patients experience AE or episodes of AE. Once acute exacerbation (AE) develops, mortality rate is 81.8% and majority of patients dies within a few month [41].

NSIP is a disease with symptoms similar to IPF. Fibrotic NSIP is the most common form of NSIP. HRCT demonstrate both ground glass and fibrotic changes. Histological features show interstitial cellular filtration and fibrosis. Due to the significant similarities of clinical and radiographic features between fibrotic NSIP and IPF, surgical biopsies is often required to distinguish these two diseases.

### SUMMARY

Provided herein are methodology, constructs, materials, kits, and the like for detecting and/or differentiating between interstitial lung diseases, such as idiopathic pulmonary fibrosis (IPF) and nonspecific interstitial pneumonia (NSIP).

In one aspect, provided is methodology for detecting IPF in a patient, comprising obtaining a subject sample and assaying for the presence of at least one of HBD-2 and HBD-4. In another aspect, the disclosure provides methodology for detecting NSIP in a patient, comprising obtaining a subject sample and assaying for the presence of at least one of HBD-9 and LL-37. In another aspect, the disclosure provides methodology for differentiating between IPF and NSIP, comprising obtaining a subject sample and assaying for the presence of at least one of HBD-2, HBD-4, HBD-9, and LL-37. A subject sample could be any sample obtained from a subject, wherein the sample is tissue, blood, and/or a bodily fluid.

In one aspect, there is provided a kit for detecting idiopathic pulmonary fibrosis (IPF), wherein said kit comprises (a) a primer or probe for detecting at least one of HBD-2 and HBD-4; and (b) an instruction manual. In one embodiment, the kit comprises a primer or probe for detecting HBD-2. In one embodiment, the kit comprises a primer or probe for detecting HBD-4. In another embodiment, the kit comprises a primer or probe for detecting HBD-2 and HBD-4.

In another aspect, provided is a kit for detecting nonspecific interstitial pneumonia (NSIP), wherein said kit comprises: (a) a primer or probe for detecting at least one of HBD-9 and LL-37; and (b) an instruction manual. In one embodiment, the kit comprises a primer or probe for detecting HBD-9. In another embodiment, the kit comprises a primer or probe for detecting LL-37. In another embodiment, the kit comprises a primer or probe for detecting HBD-9 and LL-37.

In another aspect, disclosed herein is a kit for differentiating idiopathic pulmonary fibrosis (IPF) and nonspecific interstitial pneumonia (NSIP), wherein said kit comprises:(a) a primer or probe for detecting at least one of HBD-2, HBD-4, HBD-9, and LL-37; and (b) an instruction manual.

### BRIEF DESCRIPTION OF FIGURES

FIG. 1 illustrates induction of antimicrobial peptide gene expression by IL-1β in A549 cells. A549 cells were plated and serum-starved overnight and treated with IL-1β. Twenty-four hours after IL-1β treatment, cells were harvested and RNA was extracted. Each hBD gene expression was analyzed by quantitative RT-PCR. Levels of gene expression in untreated cells were set to 1 and fold of change after IL-1β treatment was compared to control and with each other A549 cells were plated and serum-starved overnight and treated with IL-1β. Twenty-four hours after IL-1βtreatment, cells were harvested and RNA was extracted. Each hBD gene expression was analyzed by quantitative RT-PCR. Levels of gene expression in untreated cells were set to 1 and fold of change after IL-1β treatment was compared to control and with each other (*p<0.05).

FIG. 2 illustrates hBD-1 Gene Expression in Human Lung Tissue. Total RNA was extracted from 7 normal-control and 9 ILD-patient lung tissues. HBD-1 gene expression in both groups was analyzed with real-time RT-PCR (qRT-PCR). All statistical analyses were carried out using Prism 5 software (GraphPad, La Jolla, Calif.). Mann-Whitney test was performed to calculate statistical significance of the qRT-PCR (p=1.0)

FIG. 3 illustrates hBD-2 Gene Expression in Human Lung Tissue. Total RNA was extracted from 7 normal-control and 9 ILD patient lung tissues, and hBD-2 gene expression in both groups was analyzed with real-time RT-PCR (qRT-PCR). All statistical analyses were carried out using Prism 5 software (GraphPad, La Jolla, Calif.). Mann-Whitney test was performed to calculate statistical significance of the qRT-PCR (p=0.02). A. hBD-2 gene expression in ILD patients is 7.4 times higher than that in control subjects; B. Difference between ILD and control groups is 2.4 times without the outlier; C. IPF patients compared to control subjects; D. IPF patients compared to NSIP patients. (Arrow points to patient 1007033).

FIG. 4 illustrates hBD-3 Gene Expression in Human Lung Tissue. Total RNA was extracted from 7 normal-control and 9 ILD-patient lung tissues, and hBD-3 gene expression in both groups was analyzed withreal-time RT-PCR (qRT-PCR). All statistical analyses were carried out using Prism 5 software (GraphPad, La Jolla, Calif.). Mann-Whitney test was performed to calculate statistical significance of the qRT-PCR (p=0.055) (Arrow point to patient 1010-105).

FIG. 5 illustrates hBD-4 Gene Expression in Human Lung Tissue. Total RNA was extracted from 7 normal-control and 9 ILD-patient lung tissues, and hBD-4 gene expression in both groups was analyzed with real-time RT-PCR (qRT-PCR). All statistical analyses were carried out using Prism 5 software (GraphPad, La Jolla, Calif.). Mann-Whitney test was performed to calculate statistical significance of the qRT-PCR (p=0.01). HBD-4 gene expression is 4.3 times higher in ILD than that of control objects (A.) and 1.8 times higher without the outlier (B). (Arrow points to patient 100-7033).

FIG. 6 illustrates HBD-8 and 9 Gene Expression in Human Lung Tissue. Total RNA was extracted from 7 normal-control and 9 ILD-patient lung tissues, and hBD-9 gene expression in both groups was analyzed with real-time qRT-PCR. All statistical analyses were carried out using Prism 5 software (GraphPad, La Jolla, Calif.). Mann-Whitney test was performed to calculate statistical significance of the qRT-PCR (p=0.0033) (hBD-9 gene expression is 3.3 times higher in ILD than control objects. Red circle, NSIP patient lung samples; Green circle, IPF patient lung samples).

FIG. 7 illustrates HBD-5, 6 and 18 Gene Expression in Human Lung Tissue. Total RNA was extracted from 7 normal-control and 9 ILD-patient lung tissues, and hBD-5, 6 and 18 genes expression in both groups was analyzed with real-time qRT-PCR. All statistical analyses were carried out using Prism 5 software (GraphPad, La Jolla, Calif.). Mann-Whitney test was performed to calculate statistical significance of the qRT-PCR and p values are shown in the figures. (Arrow points to patient 1009092).

FIG. 8 illustrates LL-37 Gene Expression in Human Lung Tissue. Total RNA was extracted from 7 normal-control and 10 ILD-patient lung tissues, and LL-37 gene expression in both groups was analyzed with real-time qRT-PCR. All statistical analyses were carried out using Prism 5 software (GraphPad, La Jolla, Calif.). Mann-Whitney test was performed to calculate statistical significance of the qRT-PCR and p values are shown in the figures. (Arrow points to patient 1009-095-sarcoidosis).

FIG. 9 provides a comparison of IPF and NSIP, including onset age, afflicted sex, clinical symptoms, HRCT, pathology, and treatment.

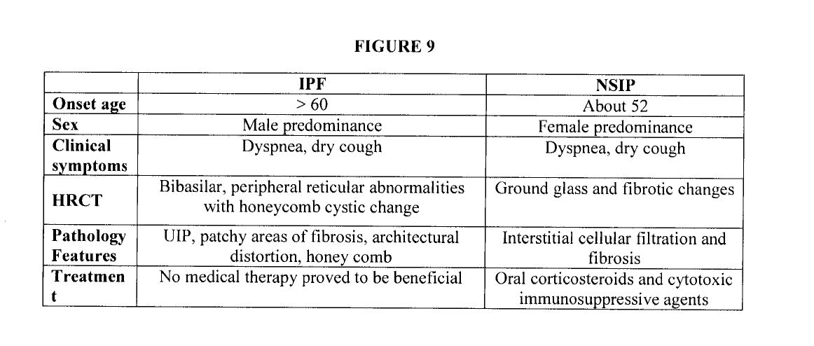

### DETAILED DESCRIPTION

Interstitial lung disease includes broad categories of diseases, including more than 100 separate disorders. Idiopathic Pulmonary Fibrosis (IPF) and Nonspecific Interstitial Pneumonia (NSIP) are subclasses of interstitial lung disease and share similar symptoms.

While IPF and NSIP share similar symptoms, their prognosis and responsiveness to treatments differ significantly. IPF is a progressive fibrotic lung disease with high morbidity and mortality and little effective medical therapy, other than transplant. In contrast, NSIP may be treated with corticosteroids. Because of significant similarities of clinical and radiographic features between IPF and NSIP, surgical biopsy may be required to distinguish between the two diseases. Sometimes, however, surgical biopsy proves ineffective for distinguishing between IPF and NSIP. For example, in 20 explanted lungs with usual interstitial pneumonia (UIP), 17 were found to have areas indistinguishable from NSIP. Katzenstein A. L., et al. Am J Surg Pathol. 2002 December; 26(12):1567-77. Additionally, sample bias may impede accurate diagnosis. Thus, differentiating between IPF and NSIP remains a challenge.

Applicants identified several biomarkers that can differentiate between interstitial lung diseases, such as IPF and NSIP. As explained in more detail below, the present inventors identified several biomarkers, such as hBD-2, 4, 9 and LL-37, that can be used for detecting and/or differentiating between IPF and NSIP.

In one embodiment, therefore, the present inventors analyzed the gene expression levels of nine human β-defensins (hBDs) and one LL-37 in whole lung tissues of seven normal subjects and ten ILD patient lungs by real time qRT-PCR. As explained below, LL37 gene expression is found to be 5.4 fold reduced in IPF patient lung, while not altered in NSIP patient lung. Contrary to LL-37, HBD-9 gene expression is 5.3 fold elevated in NSIP lung compared to control lung, and 2.9 fold higher compared to IPF lung. Additionally, hBD2 and hBD4 were found significantly elevated in IPF patients vs NSIP or control patients. Based on these results, Applicants discovered that LL-37 and hBD-2, 4 and 9 may serve as markers for the differentiation of NSIP and IPF.

These biomarkers can detect and/or differentiate between interstitial lung diseases, and the markers can be used alone and/or in conjunction with other diagnosis methods, including but not limited to symptomatic diagnosis, chest imaging, and biopsy.

All technical terms used herein are commonly used in biochemistry, molecular biology, and physiology, and can be understood by one of ordinary skill in the art. Technical terms can be found in: Molecular Cloning: A Laboratory Manual, 3rd ed., vol. 1-3, ed. Sambrook and Russell, Cold Spring Harbor Laboratory Press, Cold Spring Harbor, N.Y., 2001; Current Protocols in Molecular Biology, ed. Ausubel et al., Greene Publishing Associates and Wiley-Interscience, New York, 1988 (with periodic updates); Short Protocols in Molecular Biology: A Compendium of Methods from Current Protocols in Molecular Biology, 5th ed., vol. 1-2, ed. Ausubel et al., John Wiley & Sons, Inc., 2002; Genome Analysis: A Laboratory Manual, vol. 1-2, ed. Green et al., Cold Spring Harbor Laboratory Press, Cold Spring Harbor, N.Y., 1997. Various techniques using PCR are described in Innis et al., PCR Protocols: A Guide to Methods and Applications, Academic Press, San Diego, 1990 and in Dieffenbach and Dveksler, PCR Primer: A Laboratory Manual, 2nd ed., Cold Spring Harbor Laboratory Press, Cold Spring Harbor, N.Y., 2003.

PCR-primer pairs can be derived from known sequences by known techniques such as using computer programs intended for that purpose, Primer, Version 0.5, 1991, Whitehead Institute for Biomedical Research, Cambridge, Mass. Methods for chemical synthesis of nucleic acids are discussed, for example, in Beaucage and Caruthers, 1981, Tetra. Letts. 22: 1859-1862, and Matteucci and Caruthers, 1981 J. Am. Chem. Soc. 103: 3185. Restriction enzyme digestions, phosphorylations, ligations and transformations were done as described in Sambrook et al., Molecular Cloning: A Laboratory Manual, 2nd ed. (1989), Cold Spring Harbor Laboratory Press. Chemicals, reagents, and other materials can be obtained from suitable vendors such as Aldrich Chemicals (Milwaukee, Wis.), DIFCO Laboratories (Detroit, Mich.), Invitrogen (Gaithersburg, Md.), or Sigma Chemical Company (St. Louis, Mo.), unless otherwise specified.

Lung disease includes any condition affecting lung tissue, including but not limited to idiopathic pulmonary fibrosis (IPF), nonspecific interstitial pneumonia (NSIP), chronic obstructive lung disease, and emphysema.

Idiopathic pulmonary fibrosis (IPF) is a progressive fibrotic lung disease with high morbidity and mortality and no effective medical therapy. Common clinical features include progressive dyspnea, dry cough, and the presence of basilar ‘velcro-like’ rales on examination, which are not specific to IPF. Other pulmonary lung fibrosis diseases, such as collagen-vascular disease, chronic hypersensitivity pneumonitis, drug reactions, etc., may exhibit similar symptoms, even similar radiographic features. Diagnosis of IPF must exclude these secondary lung fibrosis diseases. The definitive diagnosis of IPF is based on surgical biopsies and pathology diagnosis. Usual interstitial pneumonia (UIP), histopathologically shown as patchy areas of fibrosis in association with areas of normal lung architecture, is the characteristic feature of IPF [38]. Once diagnosed, the median survival time is only 2.5-5 years [39] and lung transplant remains the only hope in a small minority of these patients.

Nonspecific interstitial pneumonia (NSIP) is an interstitial lung disease with symptoms similar to IPF. Fibrotic NSIP is the most common form of NSIP. HRCT demonstrate both ground glass and fibrotic changes. Histological features show interstitial cellular filtration and fibrosis. Due to the significant similarities of clinical and radiographic features between fibrotic NSIP and IPF, surgical biopsies is often required to distinguish these two diseases.

Defensins, such as human beta-defensins (HBD) and LL37, are small cationic peptides that play a role in innate immunity and participate in adaptive immunity as immune modulators. As a class, the beta-defensins may act as chemokines for cells of the adaptive immune system, especially dendritic & T cells (via CCR6 receptor), and thus may be providing a link between innate and adaptive immune systems [49]. Activation of cells by IL1-beta or TNF-alpha can lead to the increased expression of defensin peptides through receptor signaling, and through TLR signaling by pathogens. This cycle of inflammation and further recruitment of the immune cells may contribute to the progression of ILD, possibly following an acute exacerbation event for IPF, such as a viral or bacterial infection. All of these antimicrobial peptides have varying activity against bacterial, viral, and fungal pathogens.

Illustrative defensins contemplated herein include human beta defensins (HBD), such as HBD-2, HBD-4, HBD-9, and LL-37.

Biomarker refers to any molecule, such as a nucleic acid molecule, polynucleotide, oligonucleotide, probe, primer, protein, peptide, protein fragment, nucleic that can be used for detecting and/or differentiating between interstitial lung disease(s). Detection or disease differentiation may be possible with one biomarker, or may require more than biomarker. A biomarker may encompass a full length sequence, or a probe or primer designed from a portion of said sequence. A plurality of biomarkers (e.g. 3 or more biomarkers) may be referred to as a panel of biomarkers.

The term “gene” refers to a nucleic acid (e.g., DNA or RNA) sequence that comprises coding sequences necessary for the production of RNA or a polypeptide. A polypeptide can be encoded by a full-length coding sequence or by any part thereof. The term “parts thereof' when used in reference to a gene refers to fragments of that gene, particularly a fragment encoding at least a portion of a protein. The fragments may range in size from a few nucleotides to the entire gene sequence minus one nucleotide. Thus, “a nucleic acid sequence comprising at least a part of a gene” may comprise fragments of the gene or the entire gene.

“Gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated (or untranslated) sequences (5′ UTR). The sequences which are located 3′ or downstream of the coding region and which are present on the mRNA are referred to as 3′ non-translated (or untranslated) sequences (3′ UTR).

“Nucleic acid” as used herein refers to RNA or DNA that is linear or branched, single or double stranded, or a hybrid thereof. The term also encompasses RNA/DNA hybrids.

“Encoding” and “coding” refer to the process by which a gene, through the mechanisms of transcription and translation, provides information to a cell from which a series of amino acids can be assembled into a specific amino acid sequence to produce an active enzyme. Because of the degeneracy of the genetic code, certain base changes in DNA sequence do not change the amino acid sequence of a protein. It is therefore understood that modifications in the DNA sequence encoding transcription factors which do not substantially affect the functional properties of the protein are contemplated.

The term “expression,” as used herein, refers to the production of a functional end-product e.g., an mRNA or a protein.

“Heterologous gene” or “exogenous genes” refer to a gene encoding a factor that is not in its natural environment (i.e., has been altered by the hand of man). For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes may comprise gene sequences that comprise cDNA forms of a gene; the cDNA sequences may be expressed in either a sense (to produce mRNA) or anti-sense orientation (to produce an anti-sense RNA transcript that is complementary to the mRNA transcript). Heterologous genes may be distinguished from endogenous genes in that the heterologous gene sequences are typically joined to nucleotide sequences comprising regulatory elements such as promoters that are not found naturally associated with the gene for the protein encoded by the heterologous gene or with gene sequences in the chromosome, or are associated with portions of the chromosome not found in nature (e.g., genes expressed in loci where the gene is not normally expressed).

The terms “polypeptide,” “peptide” and “protein” are used interchangeably herein to refer to a polymer of amino acid residues. The terms apply to amino acid polymers in which one or more amino acid residue is an artificial chemical analog of a corresponding naturally occurring amino acid, as well as to naturally occurring amino acid polymers.

Probe or primer refers to a short oligonucleotide sequence that could be designed and synthesized, or generated as a fragment of a larger sequence. A probe or primer can be any length, such as 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length.

(A) Biomarker

In one aspect, a gene or biomarker such as hBD-2, 4, 9 and LL-37, can be used for detecting and/or differentiating between interstitial lung disease, such as IPF and NSIP. Thus, the present disclosure provides methodology, kits, and related materials for differentiating a subject with interstitial lung disease from a subject without interstitial lung disease. Additionally, the present disclosure provides methodology, biomarkers, kits, and/or related materials for detecting and/or differentiating between two interstitial lung diseases, such as IPF and NSIP.

Biomarker refers to any molecule, such as a nucleic acid molecule, polynucleotide, oligonucleotide, probe, primer, protein, peptide, protein fragment, nucleic that can be used for detecting and/or differentiating between interstitial lung disease(s). Detection or disease differentiation may be possible with one biomarker, or may require more than biomarker. As used herein, a plurality of biomarkers is referred to as a panel of biomarkers.

For example, and non-limiting, a biomarker or panel of biomarkers may be used for differentiating a subject with interstitial lung disease from a subject without interstitial lung disease. Likewise, a biomarker or panel of biomarkers may be used for differentiating between two interstitial lung diseases, such as IPF and NSIP.

As known in the art, a biomarker can be identified based on its differential expression pattern between two comparable samples. For example, and relevant to the instant disclosure, a gene that is expressed in diseased lung tissue yet absent in healthy lung tissue could constitute a biomarker for a particular lung disease. Similarly, a gene that is expressed at elevated levels in diseased lung tissue but expressed at low or moderate levels in healthy lung tissue could constitute a biomarker for a particular lung disease. Likewise, a gene that is expressed at elevated levels in disease A yet absent or expressed at moderate levels for disease B could serve as a biomarker for disease A.

Thus, a biomarker can present itself based on either (a) its detectable presence compared to no detectable presence in a control or other sample; or (b) detectable presence that is a fold change (increase or decrease) compared with a control or other sample. Therefore, and as known in the art, “detecting” includes determining presence, absence, quantity, or a combination thereof, of a biomarker(s).

In one embodiment, therefore, provided herein are biomarkers for detecting and/or differentiating between two interstitial lung diseases, such as IPF and NSIP. For example, and as described below, the present inventors found that LL37 gene expression was 5.4 fold reduced in IPF patient lung, while not altered in NSIP patient lung. Additionally, the present inventors determined that HBD-9 gene expression is 5.3 fold elevated in NSIP lung compared to control lung, and 2.9 fold higher compared to IPF lung. Additionally, hBD2 and hBD4 were found significantly elevated in IPF patients vs NSIP or control patients.

Accordingly, LL-37, hBD-2, hBD-4, and/or hBD-9 may serve as illustrative biomarkers for the differentiation of NSIP and IPF.

As noted herein, hBD-2 refers to Beta-defensin 2 (BD-2), a peptide that in humans is encoded by the DEFB4 (defensin, beta 4) gene. Harder J, et al., (July 1997). “A peptide antibiotic from human skin” Nature 387 (6636):861. Hbd-2 is synonymous with DEFB4A; BD-2; DEFB-2; DEFB102; DEFB2; DEFB4; HBD-2; SAP1, beta-defensin 4A precursor [Homo sapiens]. An illustrative HBD2 sequence is set forth in SEQ ID NO: 1.

HBD4 Sequence (Acronyms: DEFB104A; BD-4; DEFB-4; DEFB104; DEFB104B; DEFB4; hBD-4, is a beta-defensin 104 precursor [Homo sapiens]. An illustrative HBD4 sequence is set forth in SEQ ID NO: 2.

HBD9 Sequence (Acronyms: beta-defensin 109 [Homo sapiens], DEFB109P1, DEFB109, DEFB109A, DEFB109P1B, Beta-defensin 109, Defensin, beta 109, Defensin, beta 109, pseudogene 1/1B]. An illustrative HBD9 sequence is set forth in SEQ ID NO: 3.

LL-37 Sequence is synonymous with Cathelicidin and hCAP18. An illustrative LL-37 sequence is set forth in SEQ ID NO: 4.

While the sequences disclosed are exemplary, the present disclosure contemplates any differentially expressed sequence that can detect/and or differentiate interstitial lung disease.

The terms “sequence identity” and “sequence similarity” can be determined by alignment of two peptide or two nucleotide sequences using global or local alignment algorithms. Sequences may then be referred to as “substantially identical” or “essentially similar” when they share at least 70% of sequence identity over their entire length, respectively. Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or EmbossWin version 2.10.0 (using the program “needle”). Alternatively percent similarity or identity may be determined by searching against databases, using algorithm as FASTA, BLAST, etc.

The present disclosure contemplates nucleic acid molecules encoding functional proteins. As known in the art, it is understood that such proteins encompass amino acid substitutions, additions, and deletions that do not alter the function of any of the proteins.

Because many proteins are encoded by gene families, it is expected that other genes could encode proteins with similar functions as the instant polypeptides. These genes can be identified and functionally annotated by sequence comparison. A worker skilled in the art can identify a functionally related protein sequence with the aid of conventional methods such as screening cDNA libraries or genomic libraries with suitable hybridization probes. The skilled artisan knows that paralogous sequences can also be isolated with the aid of (degenerate) oligonucleotides and PCR-based methods.

Therefore, the present disclosure contemplates any nucleic acid molecule with a nucleotide sequence capable of hybridizing under stringent conditions with a sequence coding for a polypeptide equivalent to the proteins having amino acid sequences set forth as SEQ ID NO: 1-4. The term also includes sequences which cross-hybridize with the probe and primer sequences set forth in Table 2, preferably having at least about 75%, 80%, 85%, 90%, 95%, 96%, 97%, 98%, or 99% identity with the sequences shown in Table 2. The disclosure also contemplates a protein sequence that preferably is at least about 75%, 80%, 85%, 90%, 95%, 96%, 97%, 98%, or 99% identical to any of SEQ ID NO: 1-4.

“Stringent conditions” as used herein, refers to parameters with which the art is familiar, such as hybridization in 3.5.times.SSC, 1.times.Denhardt's solution, 25 mM sodium phosphate buffer (pH 7.0), 0.5% SDS, and 2 m M EDTA for 18 hours at 65.degree. C., followed by 4 washes of the filter at 65.degree. C. for 20 minutes, in 2.times.SSC, 0.1% SDS, and a final wash for up to 20 minutes in 0.5.times.SSC, 0.1% SDS, or 0.3.times.SSC and 0.1% SDS for greater stringency, and 0.1.times.SSC, 0.1% SDS for even greater stringency. Other conditions may be substituted, as long as the degree of stringency is equal to that provided herein, using a 0.5.times.SSC final wash.

Accordingly, the present disclosure comprises any nucleic acid, gene, polynucleotide, DNA, RNA, mRNA, or cDNA molecule from lung tissue, or produced synthetically, that can detect and/or differentiate interstitial lung disease. The DNA or RNA may be double-stranded or single-stranded. Single-stranded DNA may be the coding strand, also known as the sense strand, or it may be the non-coding strand, also called the anti-sense strand.

Unless otherwise indicated, all nucleotide sequences determined by sequencing were determined using an automated DNA sequencer, such as the Model 3730 from Applied Biosystems, Inc. Therefore, as is known in the art for any nucleotide sequence determined by this automated approach, any nucleotide sequence determined herein may contain some errors. Nucleotide sequences determined by automation are typically at least about 95% identical, more typically at least about 96% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence may be completely different from the amino acid sequence actually encoded by the sequenced DNA molecule, beginning at the point of such an insertion or deletion.

A “variant” is a nucleotide or amino acid sequence that deviates from the standard, or given, nucleotide or amino acid sequence of a particular gene or protein. The terms “isoform,” “isotype,” and “analog” also refer to “variant” forms of a nucleotide or an amino acid sequence. An amino acid sequence that is altered by the addition, removal, or substitution of one or more amino acids, or a change in nucleotide sequence may be considered a “variant” sequence. The variant may have “conservative” changes, wherein a substituted amino acid has similar structural or chemical properties, e.g., replacement of leucine with isoleucine. A variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software. “Variant” also may refer to a “shuffled gene,” such as those described in Maxygen-assigned patents.

(B) Biomarker Identification

Any known tool or technique may be used for identifying a biomarker. A biomarker described herein may be detected in a variety of ways.

For example, and in one embodiment, a biomarker may be detected by reverse-transcription of complementary DNAs from mRNAs obtained from a sample. In such embodiments, fluorescent dye-labeled complementary RNAs are transcribed from complementary DNAs which are then hybridized to the arrays of oligonucleotide probes. The fluorescent color generated by hybridization is read by machine, and the resultant data is processed using software, such as Agilent Feature Extraction Software (9.1).

In other embodiments, complementary DNAs are reverse-transcribed from mRNAs obtained from the sample, amplified, and simultaneously quantified by real-time PCR, thereby enabling both detection and quantification (as absolute number of copies or relative amount when normalized to DNA input or additional normalizing genes) of a specific gene product in the complementary DNA sample as well as the original mRNA sample.

In other embodiments, a biomarker may also be detected, qualitatively or quantitatively, by immunoassay procedure. The immunoassay typically includes contacting a test sample with an antibody that specifically binds to or otherwise recognizes a biomarker, and detecting the presence of a complex of the antibody bound to the biomarker in the sample. The immunoassay procedure may be selected from a wide variety of immunoassay procedures known to the art involving recognition of antibody/antigen complexes, including enzyme-linked immunosorbent assays (ELISA), radioimmunoassay (RIA), and Western blots, and use of multiplex assays, including antibody arrays, wherein several desired antibodies are placed on a support, such as a glass bead or plate, and reacted or otherwise contacted with the test sample. Such assays are well-known to the skilled artisan and are described, for example, more thoroughly in Antibodies: A Laboratory Manual (1 88) by Harlow & lane; Immunoassays: A Practical Approach, Oxford University press, Gosling, J. P. (ed.) (2001) and/or Current protocols in Molecular Biology (Ausubel et al.).

In another embodiment, microarray, DNA chip technology, or other high through-put technology may be used for identifying a biomarker. Gene expression profiling is the most common application of this technology. Hybridization between complementary nucleic acids is the basis of the technology. The microarray is a powerful tool that can give a lot of information, though analyzing a large scale of samples and interpretation of microarray can be challenging. Reproducibility and integration of large scale data have been a challenge. After data mining, results should be validated with highly reliable bio-techniques allowing for precise quantization of transcriptional abundance of identified genes.

Previously, global gene expression patterns in the IPF-AEx lung were analyzed and compared with that of stable IPF and control lung. Gene expression of α-defensins (DEFA3 and DEFA4) were found to be significantly increased in IPF-AEx lungs compared with stale IPF in the microarray data, which was confirmed by qRT-PCR. Their levels the plasma of patients with IPF-AEx were considerably higher in patients with IPF-AEx compared with control subjects or patients with stable IPF as well [50], indicating antimicrobial peptides may play a role in the pathogenesis of IPF or IPF-AEx.

Polymerase chain reaction (PCR)-based techniques may be used to detect genetic information through the specific amplification of nucleic acid sequences start with very low number of target copies. “Real time” PCR detects PCR products as they accumulate. Real time qRT-PCR gives highly se e and most accurate quantifications of gene expression with minimal handling of the samples. Microarray data often requires confirmation by qRT-PCR.

In one embodiment, real time qRT-PCR technique is used as a means to inspect whether the expression of other antimicrobial peptides are altered in ILD patient lung.

(C) Biomarker Application

The instant biomarkers can be used in many ways. For example, provided herein are diagnostic kits comprising at least one biomarker that can be used for detecting and/or differentiating an interstitial lung disease. In no way limiting, and merely for illustrative purposes, a kit may comprise a biomarker for detecting IPF or distinguishing IPF from NSIP. Such a kit may comprise other reagents, pre-fractionation spin columns, as well as an instruction manual. A biomarker may encompass a full length sequence, or a probe or primer designed from a portion of said sequence.

Additionally, an instant biomarker may be used to screen and identify compounds that may regulate biomarker expression, which may provide a path towards treatment for NSIP, for example. For example, a suitable compound may be screened based on its interaction with an instant biomarkers. By way of example, screening might include recombinantly expressing a biomarker, purifying the biomarker, and affixing the biomarker to a substrate. Additionally, a protein may recognize and cleave one or more of the instant biomarkers, therein providing a means for monitoring protein interaction via biomarker digestion.

In another application, for example, a biomarker may be used for determining genetic risk or proclivity for developing an interstitial lung disease. Thus, in one aspect, the present application provides a genetic test for IPF and/or NSIP.

In yet another embodiment, treatment can be monitored by assaying biomarker activity. For example, in the case of NSIP, before treatment, biomarker expression may be increased relative to a control, whereas during and/or post-treatment, biomarker expression could decrease.

Of course, and as known in the art, an instant biomarkers may be measured and analyzed using a variety of accepted techniques, including but not limited to western blot, RT-PCR, microarray, Southern blot, and/or northern blot.

Specific examples are presented below, which are illustrative and non-limiting.

As described below in the illustrative examples, the present inventors analyzed the gene expression levels of nine hBDs and one LL-37 in whole lung tissues of 7 normal subjects and 10 ILD patient lungs by real time qRT-PCR. As explained below, LL37 gene expression is found to be 5.4 fold reduced in IPF patient lung, while not altered in NSIP patient lung. Unlike LL-37, HBD-9 gene expression is 5.3 fold elevated in NSIP lung compared to control lung, and 2.9 fold higher compared to IPF lung. Additionally, hBD2 and hBD4 were found significantly elevated in IPF patients vs NSIP or control patients. Based on these results, LL-37 and hBD-2, 4 and 9 may serve as markers for the differentiation of NSIP and IPF.

### EXAMPLE 1

### Lung Sample Collection and Preparation

Nine (9) normal lung tissue samples from 8 individuals and 10 ILD lung tissue samples were collected from three different sources, as shown in Table 1 below.

TABLE 1
Patient Sample Table indicating type of tissue, diagnosis, pathology report,
CT scan, and outcome.

| | | | | | Outcome as |
|---|---|---|---|---|---|
| | | | Patholgy | | of March |
| Subject ID | Type of tissue | Diagnosis | report | CT scan | 2011 |
| 1007-033 | explant | IPF | IPF | ILD | died |
| | | | | | Aug. 20, 2008 due |
| | | | | | to bacterial |
| | | | | | pneumonia |
| 1007-033 | pneumonectomy | IPF | IPF | ILD | died |
| | | | | | Aug. 20, 2008 due |
| | | | | | to bacterial |
| | | | | | pneumonia |
| 1008-060 | explant | IPF | IPF | subpleural | died a couple |
| | | | | interstitial | of weeks |
| | | | | fibrosis | post- |
| | | | | | transplant |
| | | | | | most likely |
| | | | | | from |
| | | | | | pulmonary |
| | | | | | embolism |
| 1008-062 | explant | IPF | IPF | pulmonary | died 15 |
| | | | | fibrosis, | months post- |
| | | | | emphysema, | transplant |
| | | | | bronchiectasis | due to |
| | | | | | chronic |
| | | | | | rejection |
| 1009-092 | biopsy | IPF | UIP | peripheral and | worked up |
| | | | | basilar | for transplant |
| | | | | predominant | list but is too |
| | | | | interstitial | well to be |
| | | | | changes and | listed |
| | | | | honeycombing | |
| | | | | compatible with | |
| | | | | pulmonary | |
| | | | | fibrosis. | |
| 1009-097 | biopsy | NSIP | cellular and | interstitial | clinically |
| | | | fibrosing | fibrosis | stable |
| | | | interstitial | | |
| | | | pneumonia | | |
| | | | with prominent | | |
| | | | alveoloar | | |
| | | | macrophages | | |
| | | | and patchy | | |
| | | | neutrophilic | | |
| | | | infiltrates. No | | |
| | | | fibroblastic or | | |
| | | | honeycomb | | |
| | | | changes seen | | |
| 1010-101 | biopsy | NSIP | fibrosing | minimal | clinically |
| | | | interstitial | interstitial | stable |
| | | | pneumonis, | prominence at | |
| | | | favoring NSIP | the lung bases, | |
| | | | | possibly mild | |
| | | | | alectasis or mild | |
| | | | | fibrosis. No | |
| | | | | emphysema, | |
| | | | | bronchiectasis, | |
| | | | | or | |
| | | | | honeycombing | |
| | | | | changes | |
| 1010-102 | biopsy | NSIP | cellular and | pulmonary | alive, |
| | | | fibrosing non- | fibrosis | possibly with |
| | | | specific | | pulmonary |
| | | | interstitial | | hypertension |
| | | | pneumonia | | and |
| | | | with rare non- | | connective- |
| | | | caseating | | tissue |
| | | | granulomas | | disease |
| 1010-105 | biopsy | ILD | NSIP vs | none available | unknown - |
| | | | hypersensitity | | not an ALD |
| | | | pneumonitis | | clinic patient |

RNA Extraction: RNA extraction was performed using RNeasy Midi Kit, (Qiagen, Cat. 75142) according to manufacturer's instructions. Briefly, total lung tissue was broken down and ground in liquid nitrogen with pre-cooled mortar and pestle. Ground tissues (≦250 mg) were added with 4 ml of buffer RLT with beta-mercaptoehtanol, and further disrupted by passing the lysate 20 times through an 18-gauge needle fitted to an RNase-free syringe. Tissue lysate was then centrifuged for 10 min at 5000×g. Supernatant was transferred to a new 15 ml tube, 4 ml of 70% ethanol was added and mixed immediately. The mixture was transferred to an RNeasy midi column and RNA was bound to the column and washed. On-column DNase digestion was performed to remove any DNA contamination. RNA was then washed and eluted with RNase-free water and stored in −80° C. freezer. 2.0 μg of RNA from each sample was separated on a 1% Agarose gel and stained with EtBr. Only the RNA without degradation and DNA contamination was used in the following experiment.

### EXAMPLE 2

### Reverse Transcription (RT)-PCR

Real-time RT-PCR was performed to analyze antimicrobial peptide gene expression.

RT-PCR analysis was performed in a MyiQ Single Color Real-Time PCR Detection System (BioRad Laboratories) according to the manufacturer's instructions. Briefly, 2 μg of total RNA were reverse-transcribed (SuperScript™ III Reverse Transcriptase, Invitrogen). Template cDNA corresponding to 50 ng of RNA was added to a 20 μl reaction: 0.2 μM each primer and 1X iQ™ SYBR® Green Supermix (BioRad Laboratories). Samples were incubated in a 96-well PCR plate in the MyiQ Single Color Real-Time PCR Detection System. Initial denaturing: 95° C. for 3 min; 40 cycles consisting of 95° C. for 15 s, 56° C. (for hBD-1; other peptides, see Table 2) for 15 s and 72° C. for 20 s. SYBR Green fluorescence was detected at 72° C. at the end of each cycle. Melting curve profiles were produced (cooling the sample to 60° C. for 1 min and then heating slowly at 0.5° C./s up to 95° C. with continuous measurement of fluorescence) to confirm amplification of specific transcripts.

Primer sequences and amplification products are provide in Table 2 below.

TABLE 2
Primers and RT-PCR Conditions

| | | | Product |
|---|---|---|---|
| Primers | Sequences | Conditions | Size |
| hBD-1 | Forward: 5′-CCCAGTTCCTGAAATCCTGA-3′ | 40 cycles, 56° C. | 216 bp |
| | Reverse: 5′-CAGGTGCCTTGAATTTTGGT-3′ | | |
| hBD-2 | Forward: 5′-CATCAGCCATGAGGGTCTTG-3′ | 40 cycles, 58.7° C. | 199 bp |
| | Reverse: 5′-GGCTTTTTGCAGCATTTTGT-3′ | | |
| hBD-3 | Forward: 5′-AGCCTAGCAGCTATGAGGATC-3′ | 40 cycles, 56° C. | 206 bp |
| | Reverse: 5′-CTTCGGCAGCATTTTGCGCCA | | |
| hBD-4 | Forward: 5′-TTCCAGGTGTTTTTGGTGGT-3′ | 40 cycles, 57° C. | 112 bp |
| | Reverse: 5′-GAGACCACAGGTGCCAATTT-3′ | | |
| hBD-5 | Forward: 5′-TCCATCAGGTGAGTTTGCTG-3′ | 40 cycles, 57° C. | 105 bp |
| | Reverse: 5′-GTTCAGCCTGCAATTTCCAT-3′ | | |
| hBD-6 | Forward: 5′-CCCCAGCCAAGAATGCAT-3′ | 40 cycles, 55.6° C. | 78 bp |
| | Reverse: 5′-TCATTTTTCCCGCAATTGTTC-3′ | | |
| hBD-8 | Forward: 5′-CAAGTTCTACCAGCCAGGGGCAA-3′ | 40 cycles, 55.6° C. | 145 bp |
| | Reverse: 5′-TTGGTTGATGCCCCAGAGGCAG-3′ | | |
| hBD-9 | Forward: 5′-AGGTGGTTTGGGTCCTGCGGA-3′ | 40 cycles, 55.6° C. | 131 bp |
| | Reverse: 5′-TCCACCATGCTCTACAGCACTTCA-3′ | | |
| hBD-18 | Forward: 5′-TGCATTCCATCCAATGAAGA-3′ | 40 cycles, 57° C. | 181 bp |
| | Reverse: 5′-GAGGTCTCAGTTCCCCTTCC-3′ | | |
| LL-37 | Forward: 5′-CTAGAGGGAGGCAGACATGG-3′ | 40 cycles, 57° C. | 201 bp |
| | Reverse: 5′-AGGAGGCGGTAAGGTTAGC-3′ | | |

Cycle-to-cycle fluorescence emission readings were monitored and analyzed using MyiQ Software (BioRad Laboratories). Amplification products were verified by electrophoresis on a 2% agarose gel, visualized by ethidium bromide staining Relative peptide transcript levels were corrected by normalization based on the 18S transcript levels using the formula 2^{(18S cycle number (Ct)-Sample Ct)}*1000. Average gene expression levels in control objects were set to 1 and fold of gene expression change in ILD patients were compared to control objects. All statistical analyses were carried out using Prism 5 software (GraphPad, La Jolla, Calif.). Mann-Whitney test was performed to calculate statistical significance of the qRT-PCR.

### EXAMPLE 3

### Analysis of RT-PCR Results

Previously, global gene expression patterns in the IPF-AEx lung were analyzed and compared with that of stable IPF and control lung. Gene expression of α-defensins (DEFA3 and DEFA4) were found to be significantly increased in IPF-AEx lungs compared with stale IPF in the microarray data, which was confirmed by qRT-PCR. Their levels in the plasma of patients with IPF-AEx were considerably higher in patients with IPF-AEx compared with control subjects or patients with stable IPF as well [50], indicating antimicrobial peptides may play a role in the pathogenesis of IPF or IPF-AEx.

Microarray or DNA chip technology is a high through-put technology. Gene expression profiling is the most common application of this technology. Hybridization between complementary nucleic acids is the basis of the technology. While the microarray is a powerful tool that can give a lot of information, analyzing a large scale of samples and interpretation of microarray can be challenging. Reproducibility and integration of large scale data have been a challenge. After data mining, results need to be validated with highly reliable bio-techniques allowing for precise quantization of transcriptional abundance of identified genes.

Polymerase chain reaction (PCR)-based techniques are used to detect genetic information through the specific amplification of nucleic acid sequences start with very low number of target copies. “Real time” PCR detects PCR products as they accumulate. Real time qRT-PCR gives highly sensitive and most accurate quantifications of gene expression with minimal handling of the samples. Microarray data often requires confirmation by qRT-PCR. Thus we decided to use real time qRT-PCR technique as a means to inspect whether the expression of other antimicrobial peptides are altered in ILD patient lung.

In addition to the four well-studied defensins, hBD-1 to 4, 28 new defensin genes have been identified [6], including hBD-5, 6, 8, 9 and 18. Expression of defensins can be induced via NF-KB activation mainly through TLR receptor binding to microbial components, or via pro-inflammatory cytokines, such as TNF-α and IL1-β. HBD-1, 2, 8, 9 and 18 has been found to be inducible by IL1-β in human bronchial epithelial SV40-transformed cells (16HBE) [33]. Previous data has shown that over-expression of IL-1β leads to the loss of integrity of the alveolar-capillary barrier BM and destroyed lung [51].

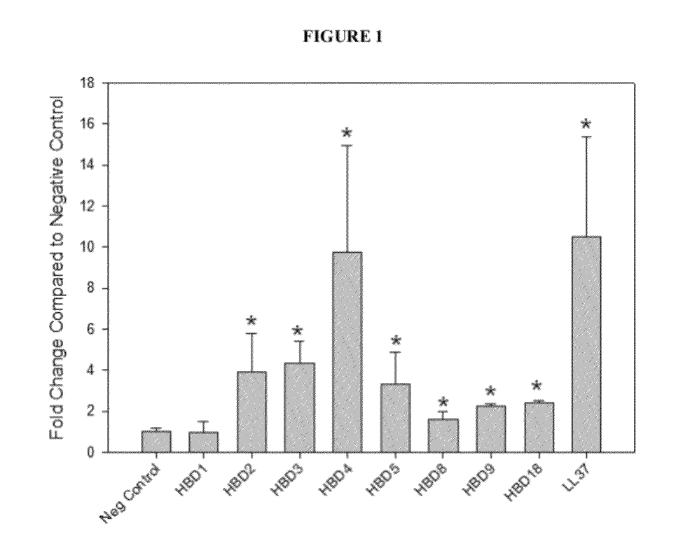

In order to see whether these antimicrobial peptides are involved in this process, it was first examined whether they are inducible in lung epithelial cells. A549 cells were plated and serum-starved overnight and treated with IL-1β. Twenty-four hours after IL-1β treatment, cells were harvested and RNA was extracted. Each hBD gene expression was analyzed by quantitative RT-PCR. Levels of gene expression in untreated cells were set to 1 and fold of change after IL-1β treatment was compared to control and with each other, as shown in FIG. 1. As shown in FIG. 1, the data demonstrate not only the gene expression of hBD-1, 2, 8, 9 and 18, but also that of hBD-3, 4, 5, and 8 are inducible by IL-1β. While a relatively new defensin, hBD-6 is not inducible by IL-1β.

### EXAMPLE 4

### hBD Gene Expression in ILD Lung

To investigate whether hBD gene expression is altered in ILD lung, total lung tissues were obtained from normal or ILD patient lungs (see Table 1).

Total RNA was extracted from these lung samples and quantitative real-time PCR was performed to quantify defensin gene expression using gene specific primers, as described above. In addition to epithelial cells, endothelial cells, macrophages, and pneumocytes also constitute the lung parenchyma. Thus, hBD-land hBD-6 gene expressions are still included even though they were found to be un-inducible by IL-1β in lung epithelial cells in vitro.

### A. HBD-1 Gene Expression is Not Altered in IPF Lung

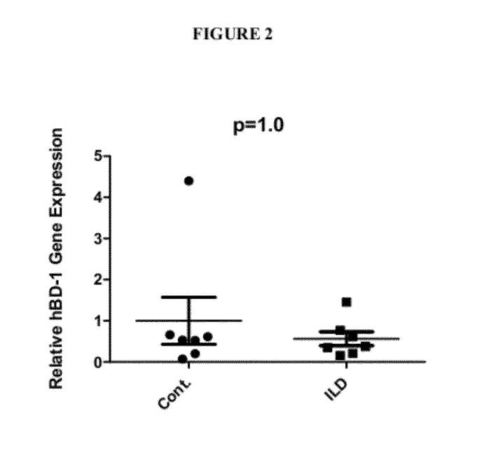

Consistent with other findings, it was determined that hBD-1 is constitutively expressed but not generally up-regulated [52, 53] (FIG. 1). This data demonstrate while hBD-1 is expressed in both normal and ILD lung, no difference was found between the two groups (FIG. 2).

### B. HBD-2 Gene Expression is Up-Regulated in IPF Lung

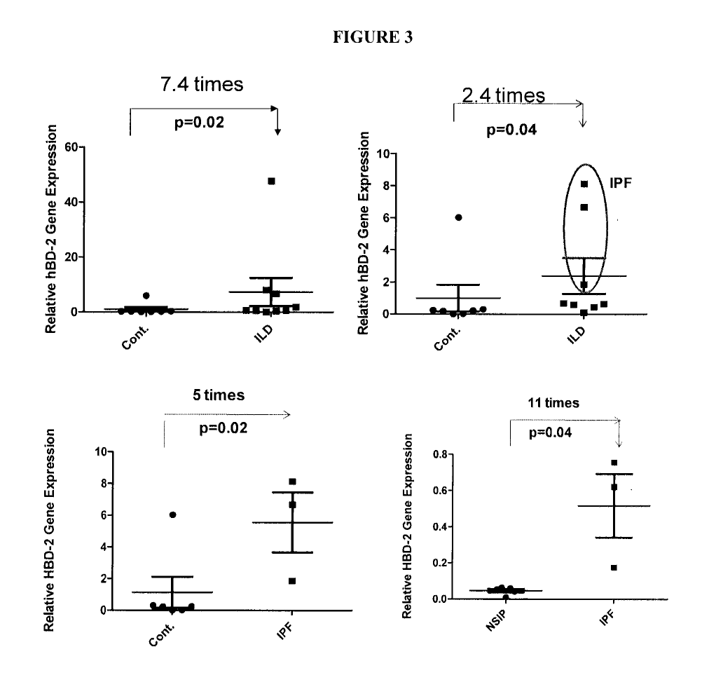

HBD-2 has been extensively studied by many groups. The gene of hBD-2 was localized to the chromosome region 8p22, where many other human defensin genes cluster [16]. HBD-2 is expressed in keratinocytes, the gingival mucosa and the tracheal epithelium [48, 58-60] and alterations of it's gene expression has been found in many diseases, such as infectious diseases, CF, and Lupus erythematosus [54]. While there is no evidence of infection in ILD patients, we found hBD-2 gene expression is significantly elevated in ILD lung tissue (7.4 fold). In close examination, patient with case number 1007033 expressed an exceptionally higher level of hBD-2 than other ILD patients. However, even if we exclude this patient, the difference (2.4 fold) between these two groups is still statistically significant (p=0.04). Thus, we believe hBD-2 gene expression is elevated in ILD patients (FIG. 3 and Table 2).

### C. HBD-3 Gene Expression is Not Altered in ILD Lung

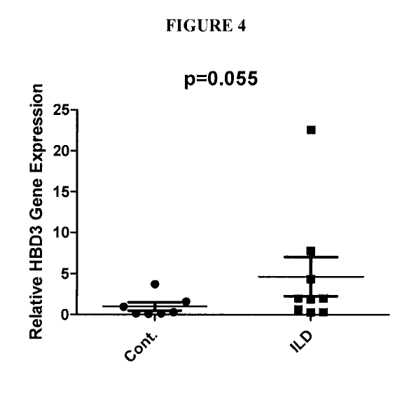

HBD-3 is an effective peptide against microbial invasion. In addition to its microbialcidal effects towards gram-negative bacteria (P. aeruginosa, E. coli) and the yeast C. albicans, hBD-3 effectively kills gram-positive bacteria such as S. pyogenes or S. aureus [9], thus, hBD-3 is a strong member of the innate immune system. Nevertheless, low hBD-3 expression was found in the epithelia of the respiratory tract, while strong hBD-3 expression was detected in keratinocytes and in tonsil tissue [55]. HBD-3 gene expression is inducible by TNF-α in keratinocytes and our data shown by IL-α in A549 lung epithelial cells (FIG. 1). However, our research found hBD-3 gene is expressed at a very low level in both normal control and ILD patient lung. HBD-3 gene expression s 4.3 fold elevated in ILD patients, but the difference is not statistically significant (p=0.055). Although our data show there is a trend that hBD-3 gene expression is elevated in ILD patient lungs, the differences between the two groups is actually largely dependent on one particular patient (case number 1010-105). If we eliminate this patient, the difference between the two groups reduced to 2.4 fold and the p value increased to 0.09. Additionally, even this patient does not have a relatively high hBD-3 gene expression comparing to other defensins. Thus, we conclude that hBD-3 is not involved in the pathogenesis of ILD (FIG. 4 and Table 3).

### D. HBD-4 Gene Eexpression is Significantly Elevated in IPF Lung

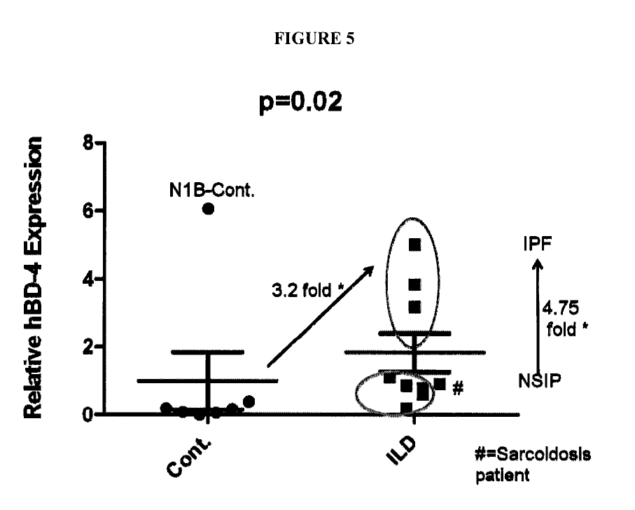

HBD-4 is a relatively new member of the defensin family. Initially it was identified by analysis of genomic sequence mapping. The hBD-4 gene maps to chromosomal region 8p23 and encodes a prepropeptide of 72 amino acids. HBD-4 gene is constitutively expressed in human lung tissue and exhibits salt sensitive antimicrobial activity against P. aeruginosa [56]. Stimulation with heat-inactivated P. aeruginosa or S. pneumoniae increased hBD-4 expression in human respiratory epithelial cells and increased hBD-4 expression is observed in lower respiratory tract infection [56]. However, contrary to previous findings that hBD-4 is not inducible in human small airway epithelial cells (SAEC 6043) by IL-1α [16], we find hBD-4 to be the most highly inducible peptide in A549 lung epithelial cells among all peptides analyzed in our study (FIG. 1). A549 cells are cancerous cells derived from lung carcinomatous tissue, while SAEC 6043 cells are normal human small airway epithelial cells. These differences may account for the differences observed in hBD-4 induction. In support of this statement, hBD-4 expression exhibits tissue specific distributions [16]. In order to see whether hBD-4 may play a role in ILD pathogenesis, we first analyzed if its' gene expression is altered in ILD lung with real time qRT-PCR as described. Our data indicate hBD-4 is constitutively expressed in human lung tissue and is significantly elevated in ILD lung (4.3 fold). In close examination of the data, hBD-4 gene expression in one IPF patient (1007-033) lung is dramatically higher than other patients, and hBD-2 gene expression in this particular patient is also significantly elevated. Thus, we suspect there might be some unknown infections present n this patient. However, if we exclude this patient in the study, the differences of hBD-4 gene expression between these two groups is reduced (1.8 fold) yet still statistically significant (p=0.02), as shown in FIG. 5 and Table 3 below.

TABLE 3
Gene Expression

| | Gene Expression | | | | | | | | | |
|---|---|---|---|---|---|---|---|---|---|---|
| Case Number | hBD-1 | hBD-2 | hBD-3 | hBD-4 | hBD-5 | hBD-6 | hBD-8 | hBD-9 | hBD-18 | LL-37 |
| N1B-Cont. | 1.51E−02 | 5.61E−01 | 2.16E−04 | 2.81E−01 | 1.43E−04 | 1.04E−05 | 1.08E−03 | 5.85E−02 | 4.00E−05 | 1.23E+00 |
| N5-Cont. | 4.61E−02 | 2.19E−02 | 6.79E−04 | 8.22E−03 | 4.24E−05 | 0.00E+00 | 3.65E−03 | 1.87E−01 | 7.76E−05 | 1.28E+00 |
| 120371-Cont | 3.90E−02 | 2.09E−02 | 8.30E−03 | 8.98E−03 | 5.20E−03 | 2.39E−03 | 1.02E−03 | 1.23E−01 | 4.97E−04 | 8.02E−01 |
| 0065664-Cont | 5.52E−03 | 9.40E−04 | 3.10E−04 | 2.96E−04 | 7.88E−06 | 0.00E+00 | 6.67E−04 | 1.43E−01 | 1.56E−05 | 2.17E−01 |
| 218081-Cont | 4.93E−02 | 4.08E−05 | 2.14E−03 | 1.84E−02 | 1.12E−03 | 6.15E−04 | 1.13E−03 | 1.82E−01 | 3.74E−04 | 6.01E−01 |
| 125484-Cont | 3.29E−01 | 2.86E−02 | 3.04E−04 | 3.39E−03 | 2.29E−04 | 6.85E−05 | 7.06E−04 | 2.33E−01 | 3.92E−03 | 9.03E−01 |
| 089451-Cont | 3.96E−02 | 1.80E−02 | 3.55E−03 | 4.04E−03 | 1.57E−03 | 2.33E−03 | 1.38E−04 | 5.04E−02 | 2.22E−04 | 4.19E−01 |
| 1008060-IPF | 2.62E−02 | 6.20E−01 | 7.15E−04 | 1.78E−01 | 4.19E−05 | 4.19E−05 | 2.20E−03 | 1.39E−01 | 2.04E−05 | 1.71E−01 |
| 1009066-IPF | 6.14E−02 | 7.56E−01 | 1.45E−03 | 2.33E−01 | 4.90E−04 | 3.00E−05 | 3.17E−03 | 2.49E−01 | 3.56E−04 | 1.95E−01 |
| 1007033-IPF | 2.85E−02 | 4.43E+00 | 4.46E−03 | 1.24E+00 | 2.11E−03 | 9.18E−04 | 3.25E−03 | 4.55E−01 | 8.11E−06 | 1.45E−01 |
| 1009092-IPF | 1.56E−02 | 1.74E−01 | 1.72E−02 | 1.48E−01 | 6.99E−02 | 3.96E−02 | 1.70E−03 | 1.88E−01 | 5.14E−03 | |
| 1008-062-IPF | | | | 4.27E−02 | | | | | | 6.41E−02 |
| 1008-095-Sarcoidosis | 1.09E−01 | 6.06E−02 | 4.17E−03 | 4.00E−02 | 1.91E−03 | 8.05E−04 | 1.80E−03 | 2.06E−01 | 4.36E−04 | 4.17E+00 |
| 1009-097-NSIP | 4.61E−02 | 4.11E−02 | 6.71E−04 | 2.86E−02 | 1.45E−04 | 2.28E−05 | 6.81E−03 | 7.24E−01 | 3.33E−05 | 1.79E+00 |
| 1010-101-NSIP | 5.78E−02 | 6.38E−02 | 4.40E−03 | 3.63E−02 | 1.95E−03 | 4.78E−04 | 9.82E−03 | 8.88E−01 | 4.72E−04 | 1.16E+00 |
| 1010-102-NSIP | 1.20E−02 | 9.93E−03 | 9.63E−03 | 9.46E−03 | 5.39E−03 | 2.50E−03 | 5.12E−03 | 7.63E−01 | 7.90E−04 | 1.05E+00 |
| 1010-105-NSIP | 9.00E−03 | 5.58E−02 | 5.00E−02 | 5.21E−02 | 8.61E−03 | 2.12E−03 | 6.57E−03 | 5.74E−01 | 3.51E−04 | 2.28E−01 |

E. HBD-8 and 9 Gene Expression is also Elevated in IPF Lung

HBD-8 is a novel defensin gene predicted by HMMER, a computational search tool. It is a peptide with a 52 amino-acid length and its gene chromosomal location is 8p23-p22, as that of hBD-1 to 9 [6]. Previous research has found hBD-8 is not constitutively expressed, but slightly inducible by IL-1β in human gingival keratinocytes [57]. Consistent with their findings, we find this gene is faintly inducible by IL-1β in lung epithelial cells (FIG. 1).

HBD-9 is a new member of the beta defensin family. It was first isolated from the ocular surface, and is constitutively expressed in human testes, placenta and peripheral blood mononuclear cells (PBMCs) [58]. Reduction of hBD-9 gene expression has been observed in ocular surface cells infected with bacteria, virus and Acanthamoeba [58], and in gingival keratinocytes infected with Candida albicans [57]. Whether this reduction is a result of the infection or the cause of the infection is unknown. Interestingly, we found hBD-9 gene expression is inducible by IL-1β (FIG. 1).

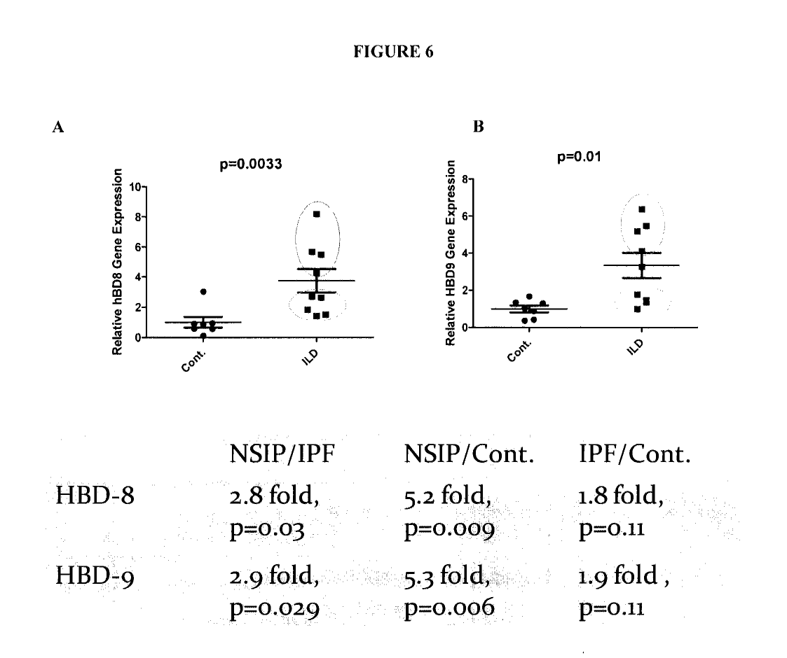

HHD-8 and 9 are new members of the defensin family. The present data indicate both defensins are inducible by IL-1β in A549 lung epithelial cells, indicating they may respond to IL-1β regulation and may be involved in inflammatory responses and cell proliferation, differentiation, and apoptosis. To investigate what role these beta-defensins play in ILD pathogenesis, we first analyzed if their gene expression level is altered in ILD lung by real time qRT-PCR. Our data show the gene expression of both defensins are elevated in IPF lung (FIG. 6). However,the level of hBD-8 gene is expressed at such a low level in both control and ILD patient groups, it is probably of no scientific significance in the disease development. HBD-9 gene expression, on the other hand, is expressed at relatively higher level in both groups and is 3.3 fold elevated in ILD lung compared to control lung.

Surprisingly, in close examine of the data, we found the elevated hBD-9 gene expression is maingly contributed by the group of NSIP patient lung, 5.3 fold of control group and 2.9 fold of IPF group. HBD-9 gene expression is not increased in IPF lung compared to control, which is consistent with previous analysis done with microarray technology [50].

### F. HBD-5, 6 and 18 Gene Expression are Not Altered in IPF Lung

The genes of hBD-5 and 6 were discovered and cloned in 2002, and found to be specifically expressed in human epididymis. Both peptides have salt sensitive antimicrobial activities, which effectively kill E. coli, but not S. aureus [59, 60]. Our data show the hBD-5 is inducible by IL-β at a very low level (FIG. 1), while hBD-6 is not inducible (data not shown).

Unlike other beta-defensins, hBD-18 is a relatively large protein with a molecular weight of 11 kDa. In addition to the cationic six-cysteine array, its C-termial extends another 68 amino acids which renders the protein slightly acidic as a whole [61].

HBD-18 gene (HBD118) is located in the β-defensin cluster on chromosome 20q11[6]. Previous study has found hBD-18 gene is only expressed in the pancreas and testis, but not in other tissues, such as the lungs [62]. Gene expression in the epididymis is regulated by androgen. The protein is present in epithelial cells of efferent ducts and most abundant in the caput epithelium, where it is present in the lumen and located on the sperm [63]. This protein has antimicrobial activity which is structure dependent and highly salt tolerant [61]. Here we found it is inducible in A549 cells by IL-1β (FIG. 1). Consistent with our data, Alekseeva et al. also found this protein is not constitutively expressed but inducible by IL-1β in human epithelial bronchial cells (16HBE) [33].

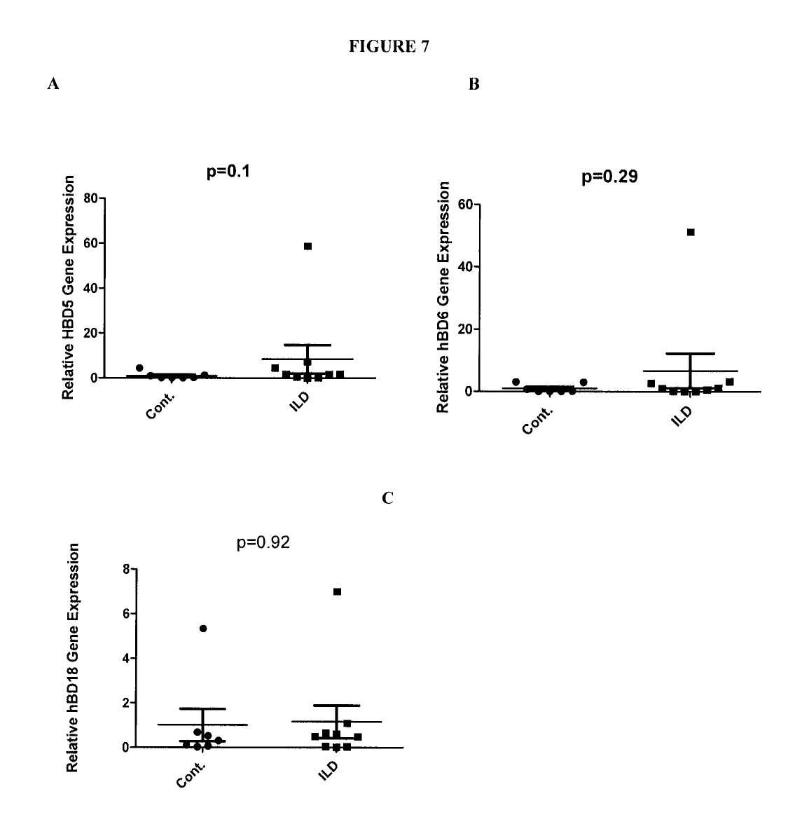

Real tune qRT-PCR show all three defensins are constitutively expressed in human lungs, but at a relatively low level and their levels of gene expression are not altered in IPF lung (FIG. 7).

### G. LL-37 Gene Expression are Not Altered in IPF Lung

The antimicrobial peptide LL-37 is the only known member of the cathelicidin family of peptides expressed in humans. In addition to its potent antimicrobial functions, LL-37 has many other tasks, such as protecting against lethal endotoxin like lipopolysacchairde (LPS), performing chemoattractant function, inhibiting neutrophil apoptosis, stimulating angiogenesis, tissue regeneration, and cytokine release (e.g. IL-8) [64]. Interestingly, LL-37 acts as a growth factor for lung cancer cells [65] and stimulate airway epithelial cell proliferation and wound closure [48, 65]. Not surprisingly, LL-37 was found to be exceedingly inducible in A549 cells by IL-1β, higher than all of the pepetides analyzed in our study. As these antimicrobial peptides are inducible in lung epithelial cells, it is possible that their expression state may be altered in the lungs of ILD. Thus we decided to analyze the gene expression of the cathelicidin L-L37 in IPF lung vs. that of the normal lung. LL-37 expression in IPF lung is 5.4 fold reduced in comparison to normal control and is not significantly changed when comparing NSIP lung vs normal control lung. However, when comparing NSIP to IPF lung, there was a 7.3 fold increase in LL-37 expression in NSIP lung vs IPF.

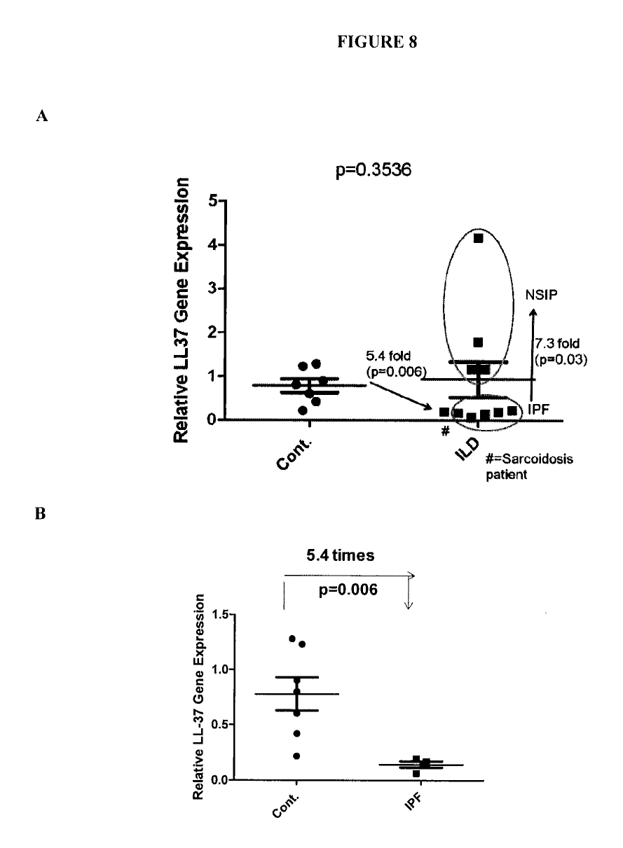

In this experiment, total RNA was extracted from 7 normal-control and 10 ILD-patient lung tissues, and LL-37 gene expression in both groups was analyzed with real-time qRT-PCR. All statistical analyses were carried out using Prism 5 software (GraphPad, La Jolla, Calif.). Mann-Whitney test was performed to calculate statistical significance of the qRT-PCR and p values are shown in the figures (FIG. 8).

SEQUENCE LISTING
1. SEQ ID NO: 1 HB2 Sequence
MRVLYLLFSFLFIFLMPLPGVFGGIGDPVTCLKSGAICHPVFCPRRYKQI
GTCGLPGTKCCKKP
2. SEQ ID NO: 2 HBD4 Sequence
MQRLVLLLAISLLLYQDLPVRSEFELDRICGYGTARCRKKCRSQEYRIGR
CPNTYACCLRKWDESLLNRTKP
3. SEQ ID NO: 3 HBD9 Sequence
MRLHLLLLILLLFSILLSPVRGGLGPAEGHCLNLFGVCRTDVCNIVEDQI
GACRRRMKCCRAWWILMPIPTPLIMSDYQEPLKPNLK
4. SEQ ID NO: 4 LL-37 Sequence
LLGDFFRKSKEKIGKEFKRIVQRIKDFLRNLVPRTES

### REFERENCES

1. Guani-Guerra, E., et al., Antimicrobial peptides: general overview and clinical implications in human health and disease. Clin Immunol. 135(1): p. 1-11.
2. Park, H. J., et al., Collagen synthesis is suppressed in dermal fibroblasts by the human antimicrobial peptide LL-37. J Invest Dermatol, 2009. 129(4): p. 843-50.
3. Hiemstra, P. S., Defensins and cathelicidins in inflammatory lung disease: beyond antimicrobial activity. Biochem Soc Trans, 2006. 34(Pt 2): p. 276-8.
4. De Smet, K. and R. Contreras, Human antimicrobial peptides: defensins, cathelicidins and histatins. Biotechnol Lett, 2005. 27(18): p. 1337-47.
5. Glukhov, E., et al., Basis for selectivity of cationic antimicrobial peptides for bacterial versus mammalian membranes. J Biol Chem, 2005. 280(40): p. 33960-7.
6. Schutte, B. C., et al., Discovery of five conserved beta-defensin gene clusters using a computational search strategy. Proc Natl Acad Sci USA, 2002. 99(4): p. 2129-33.
7. Pazgier, M., et al., Human beta-defensins. Cell Mol Life Sci, 2006. 63(11): p. 1294-313.
8. Maisetta, G., et al., In vitro bactericidal activity of human beta-defensin 3 against multidrug-resistant nosocomial strains. Antimicrob Agents Chemother, 2006. 50(2): p. 806-9.
9. Harder, J., et al., Isolation and characterization of human beta-defensin-3, a novel human inducible peptide antibiotic. J Biol Chem, 2001. 276(8): p. 5707-13.
10. Yanagi, S., et al., Significance of human beta-defensins in the epithelial lining fluid of patients with chronic lower respiratory tract infections. Clin Microbiol Infect, 2007. 13(1): p. 63-9.
11. Rizzo, A., et al., Modulation of cytokine and beta-defensin 2 expressions in human gingival fibroblasts infected with Chlamydia pneumoniae. Int Immunopharmacol, 2008. 8(9): p. 1239-47.
12. Krisanaprakornkit, S., et al., Inducible expression of human beta-defensin 2 by Fusobacterium nucleatum in oral epithelial cells: multiple signaling pathways and role of commensal bacteria in innate immunity and the epithelial barrier. Infect Immun, 2000. 68(5): p. 2907-15.
13. Dennis, D. T., et al., Tularemia as a biological weapon: medical and public health management. JAMA, 2001. 285(21): p. 2763-73.
14. Ellis, J., et al., Tularemia. Clin Microbiol Rev, 2002. 15(4): p. 631-46.
15. Jia, H. P., et al., Discovery of new human beta-defensins using a genomics-based approach. Gene, 2001. 263(1-2): p. 211-8.
16. Garcia, J. R., et al., Human beta-defensin 4: a novel inducible peptide with a specific salt-sensitive spectrum of antimicrobial activity. FASEB J, 2001. 15(10): p. 1819-21.
17. Garcia, J. R., et al., Identification of a novel, multifunctional beta-defensin (human beta-defensin 3) with specific antimicrobial activity. Its interaction with plasma membranes of Xenopus oocytes and the induction of macrophage chemoattraction. Cell Tissue Res, 2001. 306(2): p. 257-64.
18. Goldman, M. J., et al., Human beta-defensin-1 is a salt-sensitive antibiotic in lung that is inactivated in cystic fibrosis. Cell, 1997. 88(4): p. 553-60.
19. McCray, P. B., Jr. and L. Bentley, Human airway epithelia express a beta-defensin. Am J Respir Cell Mol Biol, 1997. 16(3): p. 343-9.
20. Bals, R., et al., Human beta-defensin 2 is a salt-sensitive peptide antibiotic expressed in human lung. J Clin Invest, 1998. 102(5): p. 874-80.
21. Harder, J., et al., A peptide antibiotic from human skin. Nature, 1997. 387(6636): p. 861.
22. Ishimoto, H., et al., Identification of hBD-3 in respiratory tract and serum: the increase in pneumonia. Eur Respir J, 2006. 27(2): p. 253-60.
23. Ashitani, J., et al., Elevated pleural fluid levels of defensins in patients with empyema. Chest, 1998. 113(3): p. 788-94.
24. Hiratsuka, T., et al., Identification of human beta-defensin-2 in respiratory tract and plasma and its increase in bacterial pneumonia. Biochem Biophys Res Commun, 1998. 249(3): p. 943-7.
25. Liu, P. T., et al., Cutting edge: vitamin D-mediated human antimicrobial activity against Mycobacterium tuberculosis is dependent on the induction of cathelicidin. J Immunol, 2007. 179(4): p. 2060-3.
26. Chen, C. I., et al., Beta-defensins and LL-37 in bronchoalveolar lavage fluid of patients with cystic fibrosis. J Cyst Fibros, 2004. 3(1): p. 45-50.
27. Hiemstra, P. S., S. van Wetering, and J. Stolk, Neutrophil serine proteinases and defensins in chronic obstructive pulmonary disease: effects on pulmonary epithelium. Eur Respir J, 1998. 12(5): p. 1200-8.
28. van Wetering, S., et al., Defensins: key players or bystanders in infection, injury, and repair in the lung? J Allergy Clin Immunol, 1999. 104(6): p. 1131-8.
29. Matsushita, I., et al., Genetic variants of human beta-defensin-1 and chronic obstructive pulmonary disease. Biochem Biophys Res Commun, 2002. 291(1): p. 17-22.
30. Hu, R. C., et al., Correlation of HDEFB1 polymorphism and susceptibility to chronic obstructive pulmonary disease in Chinese Han population. Chin Med J (Engl), 2004. 117(11): p. 1637-41.
31. Gudjonsson, J. E., et al., Assessment of the psoriatic transcriptome in a large sample: additional regulated genes and comparisons with in vitro models. J Invest Dermatol, 2010. 130(7): p. 1829-40.
32. Zhou, X., et al., Novel mechanisms of T-cell and dendritic cell activation revealed by profiling of psoriasis on the 63,100-element oligonucleotide array. Physiol Genomics, 2003. 13(1): p. 69-78.
33. Alekseeva, L., et al., Inducible expression of beta defensins by human respiratory epithelial cells exposed to Aspergillus fumigatus organisms. BMC Microbiol, 2009. 9: p. 33.
34. Ong, P. Y., et al., Endogenous antimicrobial peptides and skin infections in atopic dermatitis. N Engl J Med, 2002. 347(15): p. 1151-60.
35. van Wetering, S., G. S. Tjabringa, and P. S. Hiemstra, Interactions between neutrophil-derived antimicrobial peptides and airway epithelial cells. J Leukoc Biol, 2005. 77(4): p. 444-50.
36. Collard, H. R., et al., Acute exacerbations of idiopathic pulmonary fibrosis. Am J Respir Crit Care Med, 2007. 176(7): p. 636-43.
37. Mukae, H., et al., Raised plasma concentrations of alpha-defensins in patients with idiopathic pulmonary fibrosis. Thorax, 2002. 57(7): p. 623-8.
38. Harari, S. and A. Caminati, IPF: new insight on pathogenesis and treatment. Allergy, 2010. 65(5): p. 537-53.
39. Nathan, S. D., et al., Echocardiography as a predictor of pulmonary hypertension in patients with idiopathic pulmonary fibrosis. Chest, 2007. 132(4): p. 428s-428s.
40. Martinez, F. J., et al., The clinical course of patients with idiopathic pulmonary fibrosis. Ann Intern Med, 2005. 142(12 Pt 1): p. 963-7.
41. Hyzy, R., et al., Acute exacerbation of idiopathic pulmonary fibrosis. Chest, 2007. 132(5): p. 1652-8.
42. Katzenstein, A. L., et al., Usual interstitial pneumonia: histologic study of biopsy and explant specimens. Am J Surg Pathol, 2002. 26(12): p. 1567-77.
43. Johkoh, T., et al., Idiopathic interstitial pneumonias: diagnostic accuracy of thin-section CT in 129 patients. Radiology, 1999. 211(2): p. 555-60.
44. Nathan, S. D., Pulmonary hypertension in interstitial lung disease. Int J Clin Pract, 2008.
45. Mukae, H., et al., Elevated BALF concentrations of alpha-and beta-defensins in patients with pulmonary alveolar proteinosis. Respir Med, 2007. 101(4): p. 715-21.
46. Agerberth, B., et al., Antibacterial components in bronchoalveolar lavage fluid from healthy individuals and sarcoidosis patients. Am J Respir Crit Care Med, 1999. 160(1): p. 283-90.
47. Scott, M. G., et al., The human antimicrobial peptide LL-37 is a multifunctional mod0ulator of innate immune responses. J Immunol, 2002. 169(7): p. 3883-91.
48. Shaykhiev, R., et al., Human endogenous antibiotic LL-37 stimulates airway epithelial cell proliferation and wound closure. Am J Physiol Lung Cell Mol Physiol, 2005. 289(5): p. L842-8.
49. Schutte, B. C. and P. B. McCray, Jr., [beta]-defensins in lung host defense. Annu Rev Physiol, 2002. 64: p. 709-48.
50. Konishi, K., et al., Gene expression profiles of acute exacerbations of idiopathic pulmonary fibrosis. Am J Respir Crit Care Med, 2009. 180(2): p. 167-75.
51. Kolb, M., et al., Transient expression of IL-1 beta induces acute lung injury and chronic repair leading to pulmonary fibrosis. J Clin Invest, 2001. 107(12): p. 1529-36.
52. O'Neil, D. A., et al., Expression and regulation of the human beta-defensins hBD-1 and hBD-2 in intestinal epithelium. J Immunol, 1999. 163(12): p. 6718-24.
53. Eckmann, L., Defence molecules in intestinal innate immunity against bacterial infections. Curr Opin Gastroenterol, 2005. 21(2): p. 147-51.
54. Kreuter, A., et al., Expression of antimicrobial peptides in different subtypes of cutaneous lupus erythematosus. J Am Acad Dermatol.
55. Niyonsaba, F. and H. Ogawa, Protective roles of the skin against infection: implication of naturally occurring human antimicrobial agents beta-defensins, cathelicidin LL-37 and lysozyme. J Dermatol Sci, 2005. 40(3): p. 157-68.
56. Yanagi, S., et al., Isolation of human beta-defensin-4 in lung tissue and its increase in lower respiratory tract infection. Respir Res, 2005. 6: p. 130.
57. Premratanachai, P., et al., Expression and regulation of novel human beta-defensins in gingival keratinocytes. Oral Microbiol Immunol, 2004. 19(2): p. 111-7.
58. Abedin, A., et al., A novel antimicrobial peptide on the ocular surface shows decreased expression in inflammation and infection. Invest Ophthalmol Vis Sci, 2008. 49(1): p. 28-33.
59. Huang, L., et al., Production of bioactive human beta-defensin 5 and 6 in Escherichia coli by soluble fusion expression. Protein Expr Purif, 2008. 61(2): p. 168-74.
60. Yamaguchi, Y., et al., Identification of multiple novel epididymis-specific beta-defensin isoforms in humans and mice. J Immunol, 2002. 169(5): p. 2516-23.
61. Yenugu, S., et al., The androgen-regulated epididymal sperm-binding protein, human beta-defensin 118 (DEFB118) (formerly ESC42), is an antimicrobial beta-defensin. Endocrinology, 2004. 145(7): p. 3165-73.
62. Kao, C. Y., et al., ORFeome-based search of airway epithelial cell-specific novel human [beta]-defensin genes. Am J Respir Cell Mol Biol, 2003. 29(1): p. 71-80.
63. Liu, Q., et al., Primate epididymis-specific proteins: characterization of ESC42, a novel protein containing a trefoil-like motif in monkey and human. Endocrinology, 2001. 142(10): p. 4529-39.
64. Bucki, R., et al., Cathelicidin LL-37: a multitask antimicrobial peptide. Arch Immunol Ther Exp (Warsz), 2010. 58(1): p. 15-25.
65. von Haussen, J., et al., The host defence peptide LL-37/hCAP-18 is a growth factor for lung cancer cells. Lung Cancer, 2008. 59(1): p. 12-23.
66. Strider, R. M. and B. Mehrad, New mechanisms of pulmonary fibrosis. Chest, 2009. 136(5): p. 1364-70.
67. Yamada, M., et al., Dual-immunohistochemistry provides little evidence for epithelial-mesenchymal transition in pulmonary fibrosis. Histochem Cell Biol, 2008. 129(4): p. 453-62.
68. Mehrad, B., et al., Circulating peripheral blood fibrocytes in human fibrotic interstitial lung disease. Biochem Biophys Res Commun, 2007. 353(1): p. 104-8.
69. Andersson-Sjoland, A., et al., Fibrocytes are a potential source of lung fibroblasts in idiopathic pulmonary fibrosis. Int J Biochem Cell Biol, 2008. 40(10): p. 2129-40.
70. Winter, J., et al., Human beta-defensin-1, -2, and -3 exhibit opposite effects on oral squamous cell carcinoma cell proliferation. Cancer Invest. 29(3): p. 196-201.
71. Shestakova, T., et al., Immunohistochemical analysis of beta-defensin-2 expression in human lung tumors. Exp Oncol. 32(4): p. 273-6.
72. Lee, C. G., et al., Early growth response gene 1-mediated apoptosis is essential for transforming growth factor beta1-induced pulmonary fibrosis. J Exp Med, 2004. 200(3): p. 377-89.

What is claimed is:

1. A kit for detecting idiopathic pulmonary fibrosis (IPF), wherein said kit comprises:
(a) a primer or probe for detecting at least one of HBD-2 and HBD-4; and
(b) an instruction manual.

2. The kit of claim 1, wherein said kit comprises a primer or probe for detecting HBD-2.

3. The kit of claim 1, wherein said kit comprises a primer or probe for detecting HBD-4.

4. The kit of claim 1, wherein said kit comprises a primer or probe for detecting HBD-2 and HBD-4.

5. The kit of claim 1, wherein said primer or probe for at least one of HBD-2 and HBD-4 is set forth in Table 2.

6. A kit for detecting nonspecific interstitial pneumonia (NSIP), wherein said kit comprises:
(a) a primer or probe for detecting at least one of HBD-9 and LL-37; and
(b) an instruction manual.

7. The kit of claim 6, wherein said kit comprises a primer or probe for detecting HBD-9.

8. The kit of claim 6, wherein said kit comprises a primer or probe for detecting LL-37.

9. The kit of claim 6, wherein said kit comprises a primer or probe for detecting HBD-9 and LL-37.

10. The kit of claim 6, wherein said primer or probe for at least one of HBD-9 and LL-37 is set forth in Table 2.

11. A kit for differentiating idiopathic pulmonary fibrosis (IPF) and nonspecific interstitial pneumonia (NSIP), wherein said kit comprises:
(a) a primer or probe for detecting at least one of HBD-2, HBD-4, HBD-9, and LL-37; and
(b) an instruction manual.

12. The kit of claim 11, wherein a primer or probe for detecting at least one of HBD-2, HBD-4, HBD-9, and LL-37 is set forth in Table 2.
